(19) United States
(12) Patent Application Publication
(10) Pub. No.: US 2006/0247318 A1
(43) Pub. Date: Nov. 2, 2006

(54) Small molecule inhibitors of STAT3 and the uses thereof

(76) Inventors: Hui Song, Ann Arbor, MI (US); Renxiao Wang, Ann Arbor, MI (US); Shaomeng Wang, Saline, MI (US); Jiayuh Lin, West Dublin, OH (US)

Correspondence Address: MEDLEN & CARROLL, LLP;Suite 350, 101 Howard Street, San Francisco, CA, 94105, US

(21) Appl. No.: 11/361,149

(22) Filed: Feb. 24, 2006

Related U.S. Application Data
(60) Provisional application No. 60/656,597, filed on Feb. 25, 2005.

(51) Int. Cl. A61K 31/12

(52) U.S. Cl. 514/680

(57) ABSTRACT

The invention relates to small molecules which function as inhibitors of Stat3. The invention also relates to the use of these compounds for inducing cell death and sensitizing cells to the induction of cell death by anti-cancer drugs.

The present Application claims priority to U.S. Provisional Application Ser. No. 60/656,597, filed Feb. 25, 2005, which is herein incorporated by reference.

The present invention was made in part with funds under Grant No. DOD BC023370, NIH support under CA096714, and Department of Defense Breast Cancer Grant DAMD17-03-1-0508. The government may have certain rights in the invention.

### BACKGROUND OF THE INVENTION

1. Field of the Invention

This invention is in the field of medicinal chemistry. In particular, the invention relates to small molecules which function as inhibitors of Stat3. The invention also relates to the use of these compounds for inducing cell death and sensitizing cells to the induction of cell death by anti-cancer drugs.

2. Related Art

The aggressive cancer cell phenotype is the result of a variety of genetic and epigenetic alterations leading to deregulation of intracellular signaling pathways (Ponder, Nature 411:336 (2001)). The commonality for all cancer cells, however, is their failure to execute an apoptotic program, and lack of appropriate apoptosis due to defects in the normal apoptosis machinery is a hallmark of cancer (Lowe et al., Carcinogenesis 21:485 (2000)). Most of the current cancer therapies, including chemotherapeutic agents, radiation, and immunotherapy, work by indirectly inducing apoptosis in cancer cells. The inability of cancer cells to execute an apoptotic program due to defects in the normal apoptotic machinery is thus often associated with an increase in resistance to chemotherapy, radiation, or immunotherapy-induced apoptosis. Primary or acquired resistance of human cancer of different origins to current treatment protocols due to apoptosis defects is a major problem in current cancer therapy (Lowe et al., Carcinogenesis 21:485 (2000); Nicholson, Nature 407:810 (2000)). Accordingly, current and future efforts towards designing and developing new molecular target-specific anticancer therapies to improve survival and quality of life of cancer patients must include strategies that specifically target cancer cell resistance to apoptosis. In this regard, targeting crucial negative regulators that play a central role in directly inhibiting apoptosis in cancer cells represents a highly promising therapeutic strategy for new anticancer drug design.

Signal transducers and activators of transcription (STATs) are activated in response to cytokines and growth factors (Darnell et al., Science 264:1415 (1994)). JAKs, Src, and epidermal growth factor receptor (EGFR) are Stat3 upstream regulators (Bromberg et al., Mol. Cell. Biol. 18:2553 (1998); Sartor et al., Cancer Res. 57:978 (1997); Garcia et al., Oncogene 20:2499 (2001)). The main domains of Stat3 protein include the tetramerization and leucine zipper at the N-terminus, the DNA binding domain, and the SH2 transactivation domain at the carboxy-terminal end. The SH2 region is responsible for the binding of Stat3 to the tyrosine-phosphorylated receptors and for the dimerization which is necessary for DNA binding and gene expression (Zhong et al., Science 264:95(1994)). Stat3 is activated by phosphorylation at Y-705, which leads to dimer formation, nuclear translocation, recognition of Stat3-specific DNA binding elements, and activation of target gene transcription (Darnell et al., Science 264:1415 (1994); Zhong et al., Science 264:95(1994)).

The constitutive activation of Stat3 is frequently detected in breast carcinoma cell lines but not in normal breast epithelial cells (Garcia et al., Cell. Growth. Differ. 8:1267 (1997); Bowman et al., Oncogene 19:2474 (2000)). It has been reported that approximately 60 percent of breast tumors contain persistently activated Stat3 (Dechow et al., Proc. Natl. Acad. Sci. USA 101:10602 (2004)). Stat3 has been classified as a proto-oncogene because activated Stat3 can mediate oncogenic transformation in cultured cells and tumor formation in nude mice (Bromberg et al., Cell 98:295 (1999)). Stat3 may participate in oncogenesis by stimulating cell proliferation, promoting angiogenesis, and conferring resistance to apoptosis induced by conventional therapies (Catlett-Falcone et al., Curr. Opin. Oncol. 11:1 (1999); Catlett-Falcone et al., Immunity 10:105 (1999); Alas et al., Clin. Cancer Res. 9:316 (2003); Wei et al., Oncogene 22:1517 (2003)). Possible downstream targets through which Stat3 promotes oncogenesis include up-regulation of anti-apoptotic factors (Bcl-2, survivin, Mcl-1, and Bcl-X_{L}), cell-cycle regulators (cyclin D1, MEK5, and c-myc), and inducer of tumor angiogenesis (VEGF) (Bromberg et al., Cell 98:295 (1999); Wei et al., Oncogene 22:1517 (2003); Real et al., Oncogene 21:7611 (2002); Puthier et al., Eur. J. Immunol. 29:3945 (1999); Niu et al., Oncogene 21:2000 (2002); Kiuchi et al., J. Exp. Med. 189:63 (1999); Song et al., Oncogene (2004)). Activated Stat3 signaling directly contributes to malignant progression of cancer. Stat3 oncogenic function acts through the pro-survival proteins such as survivin, Mcl-1, Bcl-2, and Bcl-X_{L} and results in the prevention of apoptosis (Real et al., Oncogene 21:7611 (2002); Aoki et al., Blood 101:1535 (2003); Epling-Burnette et al., J. Clin. Invest. 107:351 (2001); Nielsen et al., Leukemia 13:735 (1999)). Blockade of Stat3 signaling inhibits cancer cell growth, demonstrating that Stat3 is essential to the survival or growth of tumor cells (Alas et al., Clin. Cancer Res. 9:316 (2003); Aoki et al., Blood 101:1535 (2003); Epling-Burnette et al., J. Clin. Invest. 107:351 (2001); Burke et al., Oncogene 20:7925 (2001); Mora et al., Cancer Res. 62:6659 (2002); Ni et al., Cancer Res. 60:1225 (2000); Rahaman et al., Oncogene 21:8404 (2002)).

Since Stat3 is frequently activated in breast cancerr (Dechow et al., Proc. Natl. Acad Sci. USA 101:10602 (2004)), it represents an attractive target for cancer therapy with the potential of inhibiting the abnormal growth of breast cancer. Peptide-based Stat3 inhibitors, which mimic the Stat3 SH2 domain complementary binding structure, were reported to successfully block Stat3 function in vitro (Turkson et al., J. Biol. Chem. 276:45443 (2001)). Attempts have also been made to inhibit Stat3 upstream regulators such as Janus kinases, especially JAK2 (Blaskovich et al., Cancer Res. 63:1270 (2003). A high-resolution X-ray three-dimensional structure of Stat3 homodimer has been disclosed (Becker et al., Nature 394:145 (1998)). There is a need for the development of small molecule inhibitors of Stat3, based on the X-ray structure of Stat3, that have high cell permeability and stability and that directly block Stat3 activity.

### SUMMARY OF THE INVENTION

It is generally accepted that the inability of cancer cells or their supporting cells to undergo apoptosis in response to genetic lesions or exposure to inducers of apoptosis (such as anticancer agents and radiation) is a major factor in the onset and progression of cancer. The induction of apoptosis in cancer cells or their supporting cells (e.g., neovascular cells in the tumor vasculature) is thought to be a universal mechanism of action for virtually all of the effective cancer therapeutic drugs or radiation therapies on the market or in practice today. One reason for the inability of a cell to undergo apoptosis is an increase in the activity of Stat3, due, at least in part, to the ability of Stat3 to up-regulate anti-apoptotic factors and/or alter cell cycle regulation.

The present invention contemplates that exposure of animals suffering from cancer to therapeutically effective amounts of drug(s) (e.g., small molecules) that decrease the function(s) of Stat3 by inhibiting the interaction between Stat3 and heterologous binding partners and/or inhibiting Stat3 homodimerization through the SH2 transactivation domain will inhibit the growth of cancer cells or supporting cells outright and/or render such cells as a population more susceptible to the cell death-inducing activity of cancer therapeutic drugs or radiation therapies. The present invention contemplates that inhibitors of Stat3 satisfy an unmet need for the treatment of multiple cancer types, either when administered as monotherapy to induce apoptosis and/or cell cycle arrest in cancer cells, or when administered in a temporal relationship with other cell death-inducing cancer therapeutic drugs or radiation therapies (combination therapies) so as to render a greater proportion of the cancer cells or supportive cells susceptible to executing the apoptosis program compared to the corresponding proportion of cells in an animal treated only with the cancer therapeutic drug or radiation therapy alone.

In certain embodiments of the invention, combination treatment of animals with a therapeutically effective amount of a compound of the present invention and a course of an anticancer agent or radiation will produce a greater tumor response and clinical benefit in such animals compared to those treated with the compound or anticancer drugs/radiation alone. Put another way, because the compounds will lower the apoptotic threshold of all cells, the proportion of cells that will successfully execute the apoptosis program in response to the apoptosis inducing activity of anticancer drugs/radiation will be increased. Alternatively, the compounds of the present invention will be used to allow administration of a lower, and therefore less toxic and more tolerable, dose of an anticancer agent and/or radiation to produce the same tumor response/clinical benefit as the conventional dose of the anticancer agent/radiation alone. Since the doses for all approved anticancer drugs and radiation treatments are known, the present invention contemplates the various combinations of them with the present compounds. Also, since the compounds of the present invention may act at least in part by inhibiting the anti-apoptotic and/or cell cycle-altering activities of Stat3, the exposure of cancer cells and supporting cells to therapeutically effective amounts of the compounds should be temporally linked to coincide with the attempts of cells to execute the apoptosis program in response to the anticancer agent or radiation therapy. Thus, in some embodiments, administering the compositions of the present invention in connection with certain temporal relationships, provides especially efficacious therapeutic practices.

The present invention relates to compounds that are useful for inhibiting the activity of Stat3 and increasing the sensitivity of cells to inducers of apoptosis and/or cell cycle arrest. In one particular embodiment, the compounds are STA-21 or a derivative, analog, prodrug, or pharmaceutically acceptable salt thereof.

In one embodiment, inhibitors of Stat3 activity are selected from the group consisting of compound 1 (STA-21), as well as the analogs identified as compounds 2 and 3.

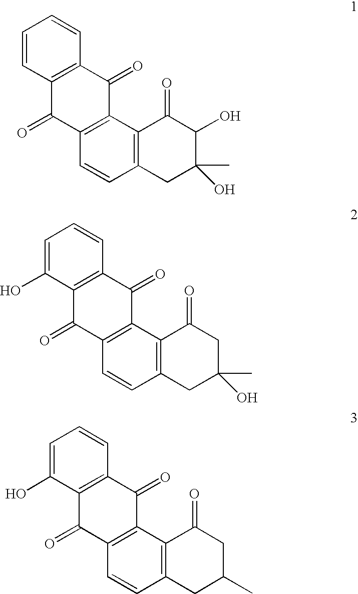

The invention relates to compounds represented by STA-21 or a derivative, analog, prodrug, or pharmaceutically acceptable salt thereof, which are inhibitors of Stat3 activity. The invention relates to the use of the compounds of the invention to inhibit the growth of cells having elevated Stat3 activity. The invention further relates to the use of the compounds of the invention to induce cell cycle arrest and/or apoptosis in cells having elevated Stat3 activity. The invention also relates to the use of the compounds of the invention for sensitizing cells to inducers of apoptosis and/or cell cycle arrest. The compounds are useful for the treatment, amelioration, or prevention of disorders associated with elevated Stat3 activity. The compounds are also useful for the treatment, amelioration, or prevention of disorders responsive to induction of apoptotic cell death, e.g., disorders characterized by dysregulation of apoptosis, including hyperproliferative diseases such as cancer and psoriasis. In certain embodiments, the compounds can be used to treat, ameliorate, or prevent cancer that is characterized by resistance to cancer therapies (e.g., those which are chemoresistant, radiation resistant, hormone resistant, and the like). In other embodiments, the compounds can be used to treat hyperproliferative diseases and other conditions characterized by elevated Stat3 activity.

The present invention provides pharmaceutical compositions comprising STA-21 or a derivative, analog, prodrug, or pharmaceutically acceptable salt thereof in a therapeutically effective amount to induce apoptosis in cells or to sensitize cells to inducers of apoptosis.

The invention further provides kits comprising STA-21 or a derivative, analog, prodrug, or pharmaceutically acceptable salt thereof and instructions for administering the compound to an animal. The kits may optionally contain other therapeutic agents, e.g., anticancer agents, apoptosis modulating agents.

### BRIEF DESCRIPTION OF THE DRAWINGS/FIGURES

The patent or application file contains at least one drawing executed in color. Copies of this patent or patent application publication with color drawing(s) will be provided by the Office upon request and payment of the necessary fee.

FIGS. 1A-1D show schematic diagrams of the modeling of structure based virtual database screening. (A) SH2 domain dimerization interface of the STAT3β protein. Structure is based on PDB entry 1BG1. The two SH2 domains are colored differently. The circled region indicates the target PTR binding bite used in the virtual screening study. (B) Predicted binding model of STA-21 to the STAT3β SH2 domain. STA-21 is rendered by the ball-and-stick model. The molecular surface of the STAT3β SH2 domain is colored with the electrostatic potentials: red for the most positively charged regions and blue for the most negatively charged regions. (C) Specific hydrogen bonds formed between the STAT3β SH2 domain and STA-21. The binding model was predicted by the DOCK program. Only the residues that form hydrogen bonds with STA-21 are shown in explicit atomic models. (D) STA-21 structure. All pictures were generated by using the Sybyl program.

FIGS. 2A-2B show the inhibition of Stat3 dependent luciferase activity in cancer cells by STA-21. (A) The clone from Caov-3 carcinoma cells stably transfected with pLucTKS3 Stat3-dependent luciferase reporter was used for initial Sat3 small molecule inhibitor screening. The cloned cells were treated with 20 μM of STA-21 as well as other small molecule compounds for 48 h, then the cells were harvested for luciferase activity analysis. (B) The clones from MDA-MB-435s cells stably transfected with pLucTKS3 Stat3-dependent luciferase reporter or SV40 luciferase reporter were treated with 20 μM of STA-21 for 48 h. Luciferase activity was measured using a Promega luciferase kit according to the manufacturer's instructions. The results were based on the averages and standard deviations from three separate experiments.

FIGS. 3A-3B show the inhibition of Stat3 DNA binding activity and Stat3-regulated anti-apoptotic factors by STA-21. (A) MDA-MB-435s cell nuclear extract was incubated with 30 μM STA-21 for 30 min at room temperature, and then incubated with r-³²P-ATP labeled consensus binding sequence for 20 min at room temperature. The reaction mixtures were resolved on 8% polyacrylamide gel. (B) The lysates from MDA-MB-468 cells treated with the indicated concentrations of STA-21 for 48 h were resolved on 10% SDS-PAGE, then immunoblotted with antibodies as indicated.

FIGS. 4A-4B show that STA-21 inhibits the survival of breast carcinoma cells with constitutive Stat3 signaling but not cells without constitutive Stat3 signaling. (A) The phosphorylation of Stat3 at Y-705 in different cell lines. (B) The cell lines were treated with STA-21 at concentrations as indicated for 48 h, and then cells were harvested and analyzed for the Sub-G1 profile that indicated apoptotic cells on a FACScan Flow Cytometer (Becton Dickinson, San Jose, Calif.). The results were based on the averages and standard deviations from three separate experiments.

FIGS. 5A-5H show that STA-21 inhibits Stat3 translocation and dimerization in breast carcinoma cells. (A) Untransfected and untreated MDA-MB-435s cells. (B and E) Cells cotransfected with pCMV-Stat3-Flag and pCMV-Stat3-HA plasmids were immunostained with anti-flag IgG-Rhodamine. (B) Untreated cells and (E) STA-21 treated cells. (C and F) Transfected cells were immunostained with anti-HA IgG-FITC. (C) Untreated cells and (F) STA-21 treated cells. (D and G) Transfected cells were co-immunostained with both anti-HA IgG-FITC and anti-flag IgG-Rhodamine. (D) Untreated cells showed bright orange color. (G) STA-21 treated cells showed weak orange and separate green and red color. (H) MDA-MB-435s cells were cotransfected with pCMV-Stat3-Flag and pCMV-Stat3-HA plasmids and were exposed to 20 μM STA-21 for 24 h, then cell lysates were immunoprecipitated with anti-HA or anti-Flag antibodies. Immunoprecipitates were resolved on 10% SDS-PAGE and then immunoblotted with anti-HA, anti-Flag or anti-Stat3 antibodies.

### DETAILED DESCRIPTION OF THE INVENTION

The present invention relates to STA-21 or derivatives, analogs, prodrugs, or pharmaceutically acceptable salts thereof, which function as inhibitors of Stat3 activity. By inhibiting Stat3, these compounds inhibit cell growth in cells having elevated Stat3 activity. These compounds also sensitize cells to inducers of apoptosis and/or cell cycle arrest and, in some instances, themselves induce apoptosis and/or cell cycle arrest. Therefore, the invention relates to methods of inhibiting cell growth, methods of sensitizing cells to inducers of apoptosis and/or cell cycle arrest and methods of inducing apoptosis and/or cell cycle arrest in cells, comprising contacting the cells with STA-21 or a derivative, analog, prodrug, or pharmaceutically acceptable salt thereof alone or in combination with an inducer of apoptosis. The invention further relates to methods of treating, ameliorating, or preventing disorders in an animal that are associated with elevated Stat3 activity or responsive to induction of apoptosis comprising administering to the animal STA-21 or a derivative, analog, prodrug, or pharmaceutically acceptable salt thereof and optionally an inducer of apoptosis. Such disorders include those characterized by a dysregulation of apoptosis and those characterized by the proliferation of cells having elevated Stat3 activity.

In one embodiment, inhibitors of Stat3 activity are selected from the group consisting of compounds 1 (STA-21), 2, and 3.

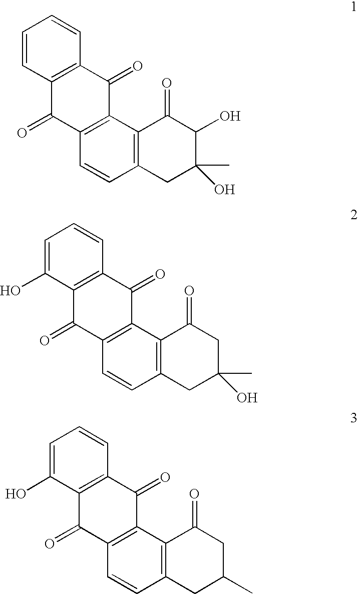

The terms “anticancer agent” and “anticancer drug,” as used herein, refer to any therapeutic agents (e.g., chemotherapeutic compounds and/or molecular therapeutic compounds), antisense therapies, radiation therapies, or surgical interventions, used in the treatment of hyperproliferative diseases such as cancer (e.g., in mammals).

The term “prodrug,” as used herein, refers to a pharmacologically inactive derivative of a parent “drug” molecule that requires biotransformation (e.g., either spontaneous or enzymatic) within the target physiological system to release, or to convert (e.g., enzymatically, mechanically, electromagnetically) the prodrug into the active drug. Prodrugs are designed to overcome problems associated with stability, toxicity, lack of specificity, or limited bioavailability. Exemplary prodrugs comprise an active drug molecule itself and a chemical masking group (e.g., a group that reversibly suppresses the activity of the drug). Some preferred prodrugs are variations or derivatives of compounds that have groups cleavable under metabolic conditions. Exemplary prodrugs become pharmaceutically active in vivo or in vitro when they undergo solvolysis under physiological conditions or undergo enzymatic degradation or other biochemical transformation (e.g., phosphorylation, hydrogenation, dehydrogenation, glycosylation). Prodrugs often offer advantages of solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common prodrugs include acid derivatives such as esters prepared by reaction of parent acids with a suitable alcohol (e.g., a lower alkanol), amides prepared by reaction of the parent acid compound with an amine, or basic groups reacted to form an acylated base derivative (e.g., a lower alkylamide).

The term “pharmaceutically acceptable salt,” as used herein, refers to any salt (e.g., obtained by reaction with an acid or a base) of a compound of the present invention that is physiologically tolerated in the target animal (e.g., a mammal). Salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, a therapeutically effective amount preferably refers to the amount of a therapeutic agent that decreases the rate of tumor growth, decreases tumor mass, decreases the number of metastases, increases time to tumor progression, or increases survival time by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “sensitize” and “sensitizing,” as used herein, refer to making, through the administration of a first agent (e.g., a compound of Formula 1), an animal or a cell within an animal more susceptible, or more responsive, to the biological effects (e.g., promotion or retardation of an aspect of cellular function including, but not limited to, cell growth, proliferation, invasion, angiogenesis, or apoptosis) of a second agent. The sensitizing effect of a first agent on a target cell can be measured as the difference in the intended biological effect (e.g., promotion or retardation of an aspect of cellular function including, but not limited to, cell growth, proliferation, invasion, angiogenesis, or apoptosis) observed upon the administration of a second agent with and without administration of the first agent. The response of the sensitized cell can be increased by at least 10%, at least 20%, at least 30%, at least 40%, at least 50%, at least 60%, at least 70%, at least 80%, at least 90%, at least 100%, at least 150%, at least 200%, at least 350%, at least 300%, at least 350%, at least 400%, at least 450%, or at least 500% over the response in the absence of the first agent.

The term “dysregulation of apoptosis,” as used herein, refers to any aberration in the ability of (e.g., predisposition) a cell to undergo cell death via apoptosis. Dysregulation of apoptosis is associated with or induced by a variety of conditions, including for example, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma or Crohn's disease), hyperproliferative disorders (e.g., tumors, B cell lymphomas, or T cell lymphomas), viral infections (e.g., herpes, papilloma, or HIV), and other conditions such as osteoarthritis and atherosclerosis. It should be noted that when the dysregulation is induced by or associated with a viral infection, the viral infection may or may not be detectable at the time dysregulation occurs or is observed. That is, viral-induced dysregulation can occur even after the disappearance of symptoms of viral infection.

The term “Stat3,” as used herein, refers to any form of Stat3 known to those of skill in the art, including, but not limited to, Stat3α and Stat3β.

The term “cells having elevated Stat3 activity,” as used herein, refers to cells in which Stat3 is constitutively activated (e.g., phosphorylated) or cells in which Stat3 is activated for a greater percentage of time or at a higher level than is found in normal (i.e., non-diseased) cells.

The term “derivative or analog thereof,” as used herein in relation to STA-21, refers to any compound which inhibits Stat3 activity and which is based on the overall structure of STA-21.

The term “hyperproliferative disease,” as used herein, refers to any condition in which a localized population of proliferating cells in an animal is not governed by the usual limitations of normal growth. Examples of hyperproliferative disorders include tumors, neoplasms, lymphomas and the like and non-cancer disorders such as autoimmune diseases (e.g., psoriasis). A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The pathological growth of activated lymphoid cells often results in an autoimmune disorder or a chronic inflammatory condition. As used herein, the term “autoimmune disorder” refers to any condition in which an organism produces antibodies or immune cells which recognize the organism's own molecules, cells or tissues. Non-limiting examples of autoimmune disorders include autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjögren's syndrome, systemic lupus erythematosus, type 1 diabetes, ulcerative colitis, vitiligo, and the like.

The term “neoplastic disease,” as used herein, refers to any abnormal growth of cells being either benign (non-cancerous) or malignant (cancerous).

The term “anti-neoplastic agent,” as used herein, refers to any compound that retards the proliferation, growth, or spread of a targeted (e.g., malignant) neoplasm.

The terms “prevent,” “preventing,” and “prevention,” as used herein, refer to a decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

The term “apoptosis modulating agents,” as used herein, refers to agents which are involved in modulating (e.g., inhibiting, decreasing, increasing, promoting) apoptosis. Examples of apoptosis modulating agents include proteins which comprise a death domain such as, but not limited to, Fas/CD95, TRAMP, TNF RI, DR1, DR2, DR3, DR4, DR5, DR6, FADD, and RIP. Other examples of apoptotic modulating agents include, but are not limited to, TNFα, Fas ligand, antibodies to Fas/CD95 and other TNF family receptors, TRAIL, antibodies to TRAILR1 or TRAILR2, Bcl-2, p53, BAX, BAD, Akt, CAD, P13 kinase, PP1, and caspase proteins. Modulating agents broadly include agonists and antagonists of TNF family receptors and TNF family ligands. Apoptosis modulating agents may be soluble or membrane bound (e.g. ligand or receptor). Preferred apoptosis modulating agents are inducers of apoptosis, such as TNF or a TNF-related ligand, particularly a TRAMP ligand, a Fas/CD95 ligand, a TNFR-1 ligand, or TRAIL.

The inhibitors of Stat3 activity of the present invention are STA-21 or a derivative, analog, prodrug, or pharmaceutically acceptable salt thereof.

Certain of the compounds of the present invention may exist as stereoisomers including optical isomers. The invention includes all stereoisomers and both the racemic mixtures of such stereoisomers as well as the individual enantiomers that may be separated according to methods that are well known to those of skill in the art.

The compounds of the present invention may be prepared using routine methods well known in the art.

An important aspect of the present invention is that STA-21 or derivatives, analogs, prodrugs, or pharmaceutically acceptable salts thereof can inhibit cell growth, at least in part by inducing cell cycle arrest and/or apoptosis, and can also potentiate the induction of cell cycle arrest and/or apoptosis in response to apoptosis induction signals. Therefore, it is contemplated that these compounds sensitize cells to inducers of apoptosis, including cells that are resistant to such inducers. The inhibitors of Stat3 activity of the present invention can be used to induce apoptosis in any disorder that can be treated, ameliorated, or prevented by the induction of apoptosis. In one embodiment, the inhibitors can be used to induce apoptosis in cells having elevated Stat3 activity.

In another embodiment, the invention pertains to modulating an apoptosis associated state which is associated with one or more apoptosis modulating agents. Examples of apoptosis modulating agents include, but are not limited to, Fas/CD95, TRAMP, TNF RI, DR1, DR2, DR3, DR4, DR5, DR6, FADD, RIP, TNFα, Fas ligand, TRAIL, antibodies to TRAILR1 or TRAILR2, Bcl-2, p53, BAX, BAD, Akt, CAD, P13 kinase, PP1, and caspase proteins. Other agents involved in the initiation, decision and degradation phase of apoptosis are also included. Examples of apoptosis modulating agents include agents, the activity, presence, or change in concentration of which, can modulate apoptosis in a subject. Preferred apoptosis modulating agents are inducers of apoptosis, such as TNF or a TNF-related ligand, particularly a TRAMP ligand, a Fas/CD95 ligand, a TNFR-1 ligand, or TRAIL.

In some embodiments, the compositions and methods of the present invention are used to treat diseased cells, tissues, organs, or pathological conditions and/or disease states in an animal (e.g., a mammalian subject including, but not limited to, humans and veterinary animals). In this regard, various diseases and pathologies are amenable to treatment or prophylaxis using the present methods and compositions. A non-limiting exemplary list of these diseases and conditions includes, but is not limited to, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like, T and B cell mediated autoimmune diseases; inflammatory diseases; infections; hyperproliferative diseases; AIDS; degenerative conditions, vascular diseases, and the like. In one embodiment, the cancer is breast cancer or ovarian cancer. In some embodiments, the cancer cells being treated are metastatic. In other embodiments, the cancer cells being treated are resistant to anticancer agents.

In some embodiments, infections suitable for treatment with the compositions and methods of the present invention include, but are not limited to, infections caused by viruses, bacteria, fungi, mycoplasma, prions, and the like.

Some embodiments of the present invention provide methods for administering an effective amount of a compound of the present invention and at least one additional therapeutic agent (including, but not limited to, chemotherapeutic antineoplastics, apoptosis modulating agents, antimicrobials, antivirals, antifungals, and anti-inflammatory agents) and/or therapeutic technique (e.g., surgical intervention, and/or radiotherapies).

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In preferred embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAILR1 or TRAILR2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of Formula I and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., Emeryville, |
| (des-alanyl-1, serine-125 human interleukin-2) | | CA |
| Alemtuzumab | Campath | Millennium and ILEX |
| (IgG1κ anti CD52 antibody) | | Partners, LP, Cambridge, |
| | | MA |
| Alitretinoin | Panretin | Ligand Pharmaceuticals, Inc., |
| (9-cis-retinoic acid) | | San Diego CA |
| Allopurinol | Zyloprim | GlaxoSmithKline, Research |
| (1,5-dihydro-4H-pyrazolo[3,4-d]pyrimidin-4- | | Triangle Park, NC |
| one monosodium salt) | | |
| Altretamine | Hexalen | US Bioscience, West |
| (N,N,N′,N′,N″,N″,-hexamethyl-1,3,5-triazine- | | Conshohocken, PA |
| 2,4,6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a,a,a′,a′- | | Pharmaceuticals, LP, |
| tetramethyl-5-(1H-1,2,4-triazol-1-ylmethyl)) | | Wilmington, DE |
| Arsenic trioxide | Trisenox | Cell Therapeutic, Inc., |
| | | Seattle, WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, Corp., |
| (lyophilized preparation of an attenuated strain | | Durham, NC |
| of Mycobacterium bovis (Bacillus Calmette- | | |
| Gukin [BCG], substrain Montreal) | | |
| bexarotene capsules | Targretin | Ligand Pharmaceuticals |
| (4-[1-(5,6,7,8-tetrahydro-3,5,5,8,8- | | |
| pentamethyl-2-napthalenyl) ethenyl] benzoic | | |
| acid) | | |
| bexarotene gel | Targretin | Ligand Pharmaceuticals |
| Bleomycin | Blenoxane | Bristol-Myers Squibb Co., |
| (cytotoxic glycopeptide antibiotics produced | | NY, NY |
| by Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2−)-0,0′]-,(SP-4-2)) | | |
| Carmustine | BCNU, | Bristol-Myers Squibb |
| (1,3-bis(2-chloroethyl)-1-nitrosourea) | BiCNU | |
| Carmustine with Polifeprosan 20 Implant | Gliadel | Guilford Pharmaceuticals, |
| | Wafer | Inc., Baltimore, MD |
| Celecoxib | Celebrex | Searle Pharmaceuticals, |
| (as 4-[5-(4-methylphenyl)-3-(trifluoromethyl)- | | England |
| 1H-pyrazol-1-yl] | | |
| benzenesulfonamide) | | |
| Chlorambucil | Leukeran | GlaxoSmithKline |
| (4-[bis(2chlorethyl)amino]benzenebutanoic | | |
| acid) | | |
| Cisplatin | Platinol | Bristol-Myers Squibb |
| (PtCl₂H₆N₂) | | |
| Cladribine | Leustatin, | R.W. Johnson |
| (2-chloro-2′-deoxy-b-D-adenosine) | 2-CdA | Pharmaceutical Research |
| | | Institute, Raritan, NJ |
| Cyclophosphamide | Cytoxan, | Bristol-Myers Squibb |
| (2-[bis(2-chloroethyl)amino] tetrahydro-2H- | Neosar | |
| 13,2-oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, Inc., |
| | | San Diego, CA |
| Dacarbazine | DTIC- | Bayer AG, Leverkusen, |
| (5-(3,3-dimethyl-1-triazeno)-imidazole-4- | Dome | Germany |
| carboxamide (DTIC)) | | |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., Thousand Oaks, |
| (recombinant peptide) | | CA |
| daunorubicin liposomal | DanuoXome | Nexstar Pharmaceuticals, |
| ((8S-cis)-8-acetyl-10-[(3-amino-2,3,6-trideoxy- | | Inc., Boulder, CO |
| a-L-lyxo-hexopyranosyl)oxy]-7,8,9,10- | | |
| tetrahydro-6,8,11-trihydroxy-1-methoxy-5,12- | | |
| naphthacenedione hydrochloride) | | |
| Daunorubicin HCl, daunomycin | Cerubidine | Wyeth Ayerst, Madison, NJ |
| ((1S,3S)-3-Acetyl-1,2,3,4,6,11-hexahydro- | | |
| 3,5,12-trihydroxy-10-methoxy-6,11-dioxo-1- | | |
| naphthacenyl 3-amino-2,3,6-trideoxy-(alpha)- | | |
| L-lyxo-hexopyranoside hydrochloride) | | |
| Denileukin diftitox | Ontak | Seragen, Inc., Hopkinton, |
| (recombinant peptide) | | MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis Pharmaceuticals, |
| ((2R,3S)-N-carboxy-3-phenylisoserine, N-tert- | | Inc., Bridgewater, NJ |
| butyl ester, 13-ester with 5b-20-epoxy- | | |
| 12a,4,7b,10b,13a-hexahydroxytax-11-en-9- | | |
| one 4-acetate 2-benzoate, trihydrate) | | |
| Doxorubicin HCl | Adriamycin, | Pharmacia & Upjohn |
| (8S,10S)-10-[(3-amino-2,3,6-trideoxy-a-L- | Rubex | Company |
| lyxo-hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin | Pharmacia & Upjohn |
| | PFS | Company |
| | Intravenous | |
| | injection | |
| doxorubicin liposomal | Doxil | Sequus Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo Alto, CA |
| | injection | |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-,3- | | Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine(intraarterial) | FUDR | Roche |
| (2′-deoxy-5-fluorouridine) | | |
| Fludarabine | Fludara | Berlex Laboratories, Inc., |
| (fluorinated nucleotide analog of the antiviral | | Cedar Knolls, NJ |
| agent vidarabine, 9-b-D- | | |
| arabinofuranosyladenine (ara-A)) | | |
| Fluorouracil, 5-FU | Adrucil | ICN Pharmaceuticals, Inc., |
| (5-fluoro-2,4(1H,3H)-pyrimidinedione) | | Humacao, Puerto Rico |
| Fulvestrant | Faslodex | IPR Pharmaceuticals, |
| (7-alpha-[9-(4,4,5,5,5-penta | | Guayama, Puerto Rico |
| fluoropentylsulphinyl) nonyl]estra-1,3,5-(10)- | | |
| triene-3,17-beta-diol) | | |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex | AstraZeneca Pharmaceuticals |
| (acetate salt of [D-Ser(But)⁶,Azgly¹⁰]LHRH; | Implant | |
| pyro-Glu-His-Trp-Ser-Tyr-D-Ser(But)-Leu- | | |
| Arg-Pro-Azgly-NH2 acetate [C₅₉H₈₄N₁₈O₁₄. | | |
| (C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal | | |
| antibody Ibritumomab and the linker-chelator | | |
| tiuxetan [N-[2-bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3- | | Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La Roche, Inc., |
| (recombinant peptide) | | Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperidinopiperidino) | | Company |
| carbonyloxy]-1H-pyrano[3′,4′: | | |
| 6,7] indolizino[1,2-b] quinoline-3,14(4H,12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., Seattle, WA |
| (L-Glutamic acid, N[4[[(2amino-5-formyl- | Leucovorin | |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((−)-(S)-2,3,5,6-tetrahydro-6-phenylimidazo | | Foundation, Titusville, NJ |
| [2,1-b] thiazole monohydrochloride | | |
| C₁₁H₁₂N₂S.HCl) | | |
| Lomustine | CeeNU | Bristol-Myers Squibb |
| (1-(2-chloro-ethyl)-3-cyclohexyl-1- | | |
| nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6H-purine-6-thione | | |
| monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way Exton, |
| (9-methoxy-7H-furo[3,2-g][1]-benzopyran-7- | | Pa |
| one) | | |
| Mitomycin C | Mutamycin | Bristol-Myers Squibb |
| mitomycin C | Mitozytrex | SuperGen, Inc., Dublin, CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin- | Organon, Inc., West Orange, |
| | 50 | NJ |
| Nofetumomab | Verluma | Boehringer Ingelheim |
| | | Pharma KG, Germany |
| Oprelvekin | Neumega | Genetics Institute, Inc., |
| (IL-11) | | Alexandria, VA |
| Oxaliplatin | Eloxatin | Sanofi Synthelabo, Inc., NY, |
| (cis-[(1R,2R)-1,2-cyclohexanediamine-N,N′] | | NY |
| [oxalato(2-)-O,O′] platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10-diacetate 2- | | |
| benzoate 13-ester with (2R,3S)-N-benzoyl-3- | | |
| phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon Pharmaceuticals, Inc., |
| ((monomethoxypolyethylene glycol | (Pegademase | Bridgewater, NJ |
| succinimidyl) 11-17-adenosine deaminase) | Bovine) | |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis Pharmaceutical |
| | | Co., Rockville, MD |
| Pipobroman | Vercyte | Abbott Laboratories, Abbott |
| | | Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, Inc., |
| | | Vancouver, |
| | | Canada |
| Procarbazine | Matulane | Sigma Tau Pharmaceuticals, |
| (N-isopropyl-μ-(2-methylhydrazino)-p- | | Inc., Gaithersburg, MD |
| toluamide monohydrochloride) | | |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, Inc., |
| (recombinant peptide) | | |
| Rituximab | Rituxan | Genentech, Inc., South San |
| (recombinant anti-CD20 antibody) | | Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]- | | |
| a(and b)-D-glucopyranose and 220 mg citric | | |
| acid anhydrous) | | |
| Talc | Sclerosol | Bryan, Corp., Woburn, MA |
| (Mg₃Si₄O₁₀(OH)₂) | | |
| Tamoxifen | Nolvadex | AstraZeneca Pharmaceuticals |
| ((Z)2-[4-(1,2-diphenyl-1-butenyl) phenoxy]-N, | | |
| N-dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]- | | |
| as-tetrazine-8-carboxamide) | | |
| teniposide, VM-26 | Vumon | Bristol-Myers Squibb |
| (4′-demethylepipodophyllotoxin 9-[4,6-0-(R)- | | |
| 2-thenylidene-(beta)-D-glucopyranoside]) | | |
| Testolactone | Teslac | Bristol-Myers Squibb |
| (13-hydroxy-3-oxo-13,17-secoandrosta-1,4- | | |
| dien-17-oic acid [dgr]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex Corporation |
| (Aziridine, 1,1′,1″-phosphinothioylidynetris-, | | |
| or Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′,4′:6,7] indolizino | | |
| [1,2-b] quinoline-3,14-(4H,12H)-dione | | |
| monohydrochloride) | | |
| Toremifene | Fareston | Roberts Pharmaceutical |
| (2-(p-[(Z)-4-chloro-1,2-diphenyl-1-butenyl]- | | Corp., Eatontown, NJ |
| phenoxy)-N,N-dimethylethylamine citrate | | |
| (1:1)) | | |
| Tositumomab, I 131 Tositumomab | Bexxar | Corixa Corp., Seattle, WA |
| (recombinant murine immunotherapeutic | | |
| monoclonal IgG₂ₐ lambda anti-CD20 antibody | | |
| (I 131 is a radioimmunotherapeutic antibody)) | | |
| Trastuzumab | Herceptin | Genentech, Inc |
| (recombinant monoclonal IgG₁ kappa anti- | | |
| HER2 antibody) | | |
| Tretinoin, ATRA | Vesanoid | Roche |
| (all-trans retinoic acid) | | |
| Uracil Mustard | Uracil | Roberts Labs |
| | Mustard | |
| | Capsules | |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2- | | |
| oxoethyl pentanoate) | | |
| Vinblastine, Leurocristine | Velban | Eli Lilly |
| (C₄₆H₅₆N₄O₁₀.H₂SO₄) | | |
| Vincristine | Oncovin | Eli Lilly |
| (C₄₆H₅₆N₄O₁₀.H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

The present invention provides methods for administering a compound of the invention with radiation therapy. The invention is not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The animal may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. The dose of radiation is preferably fractionated for maximal target cell exposure and reduced toxicity.

The total dose of radiation administered to an animal preferably is about 0.01 Gray (Gy) to about 100 Gy. More preferably, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), preferably 1-2 Gy (e.g., 1.5-2 Gy). The daily dose of radiation should be sufficient to induce destruction of the targeted cells. If stretched over a period, radiation preferably is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. Preferably, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, STA-21 or a derivative, analog, prodrug, or pharmaceutically acceptable salt thereof and one or more therapeutic agents or anticancer agents are administered to an animal under one or more of the following conditions: at different periodicities, at different durations, at different concentrations, by different administration routes, etc. In some embodiments, the compound is administered prior to the therapeutic or anticancer agent, e.g., 0.5, 1, 2, 3, 4, 5, 10, 12, or 18 hours, 1, 2, 3, 4, 5, or 6 days, or 1, 2, 3, or 4 weeks prior to the administration of the therapeutic or anticancer agent. In some embodiments, the compound is administered after the therapeutic or anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, the compound and the therapeutic or anticancer agent are administered concurrently but on different schedules, e.g., the compound is administered daily while the therapeutic or anticancer agent is administered once a week, once every two weeks, once every three weeks, or once every four weeks. In other embodiments, the compound is administered once a week while the therapeutic or anticancer agent is administered daily, once a week, once every two weeks, once every three weeks, or once every four weeks.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. Preferably, about 0.01 to about 10 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, and most preferably, from about 0.01 to about 5 mg/kg.

The unit oral dose may comprise from about 0.01 to about 1000 mg, preferably about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a preferred embodiment, the compound is present at a concentration of about 0.07-1.0 mg/ml, more preferably, about 0.1-0.5 mg/ml, most preferably, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. Preferably, the preparations, particularly those preparations which can be administered orally or topically and which can be used for the preferred type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by injection, topically or orally, contain from about 0.01 to 99 percent, preferably from about 0.25 to 75 percent of active compound(s), together with the excipient.

The pharmaceutical compositions of the invention may be administered to any animal which may experience the beneficial effects of the compounds of the invention. Foremost among such animals are mammals, e.g., humans, although the invention is not intended to be so limited. Other animals include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight.

Lotions may be conveniently prepared by dissolving the active ingredient, in a suitable high molecular weight alcohol such as propylene glycol or polyethylene glycol.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

### Structure-Based Virtual Screening for Stat3 Inhibitors

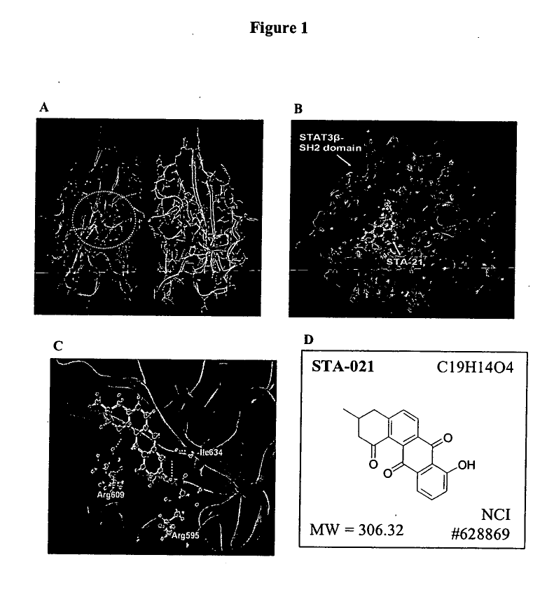

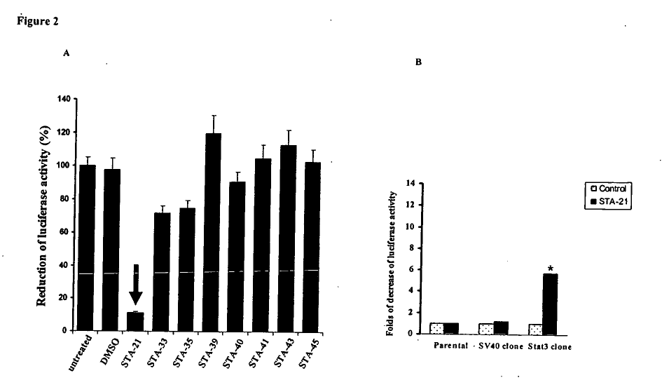

The three-dimensional structure of STAT3β homodimer shows that the dimerization of Stat3β occurs between two SH2 domains (FIG. 1A) (Becker et al., Nature 394:145 (1998); Berman et al., Nucleic Acids Res. 28:235 (2000)). These two SH2 domains are hinged together by a loop segment (Ala702-Phe716) from each monomer. The phosphoryl tyrosine (Y-705) critical for Stat3β's biological function is located in this loop segment, and it binds together with four adjacent amino acid residues to a cavity on the SH2 domain of the other monomer.

In order to identify small molecules that may disturb the dimerization process of Stat3β, the crystal structure of Stat3β solved at 2.25 Å resolution (entry 1BG1 in the Protein Data Bank) was employed in this study. The three-dimensional structure of Stat3β homodimer bound to DNA was directly obtained from Dr. C. W. Muller. The chemical databases in the virtual screening effort included the National Cancer Institute (NCI) database, the Merck Index, the Aldrich-Sigma catalogue, and the Ryan Scientific catalogue. Collectively, these four databases offered a collection of nearly 429,000 organic compounds. The publicly available NCI database itself provides three-dimensional structural models for the compounds in its contents. The other three chemical catalogues only provide two-dimensional chemical structures of their compounds. To tackle this problem, the CORINA program (version 2.6, Molecular Networks GmbH Inc., Erlangen, Germany) was applied to generate the three-dimensional structural models for the compounds in these three databases. The standard setting in the CORINA program was adopted in this process.

The molecular docking program DOCK (version 4.0) (Ewing et al., J. Comput. Aided Mol. Des. 15:411 (2001)) was used to perform the virtual screening. The binding cavity on the Stat3β SH2 domain was defined as the targeted region for docking molecules. The Sybyl software (version 6.9, Tripos Inc, St. Louise, Mo.) was used to assign the standard AMBER (refers to a set of molecular mechanical force fields for the simulation of biomolecules) atomic partial charges on the Stat3β protein and the Gasteiger-Huckel atomic partial charges on each ligand molecule to be docked. Each molecule in the databases with a molecular weight falling between 200 to 700 was docked into the targeted binding site. The top 10% scored compounds from each database, as selected by the DOCK program, were extracted and combined together to give a total of nearly 35,000 compounds. Based on the binding models of these compounds suggested by the DOCK program, the X-Score program (version 1.1) (Wang et al., J. Comput. Aided Mol. Des. 16:11 (2002)) was further applied to get a better estimation of the binding affinities of these compounds. The pre-selected 35,000 compounds were then re-ranked by the binding affinities predicted by the X-Score program. Of the best-scored 200 compounds selected by the X-Score program from this pool, 100 compounds were obtained from NCI or purchased from Aldrich-Sigma Corp. (St. Louis, Mo.) and Ryan Scientific Inc. (Isle of Palms, S.C.).

All of the obtained compounds were screened using an in vitro cell luciferase assay (see below). Of the 100 compounds tested, the most promising compound was STA-21 obtained from NCI (NCI No: 628869). STA-21 is a natural product extract and an angucycline antibiotics tetrangomycin analog with a mass of 306 Da (C₁₉H₁₄O₄) (FIG. 1D). The original predicted binding mode of STA-21 was given by the DOCK program. It was then refined by a structural optimization within the constraints of the binding site using the AMBER force field implemented in the Sybyl software 6.9 version. The refined model is shown in FIGS. 1B and 1C. This model predicts that STA-21 binds at the same site where the PTR residue binds, and it may form specific hydrogen bonds with several nearby residues, including Arg595, Arg609, and Ile634 (FIG. 1C). Based on this binding model, the X-Score program predicted that STA-21 has a binding affinity to the Stat3β SH2 domain of K_{d}≈3 μM.

### EXAMPLE 2

### Effect of STA-21 on Stat3 Transcriptional Activity

The 100 selected inhibitors were evaluated using a Stat3 luciferase reporter system. Both MDA-MB-435s breast carcinoma cells and Caov-3 ovarian carcinoma cells express constitutively activated Stat3 (Song et al., Int. J. Oncol. 24:1017 (2004); Song et al., Biochem. Biophys. Res. Commun. 314:143 (2004)). Cloned cells were established from these two cell lines by stable transfection of a Stat3-dependent luciferase reporter, pLucTKS3 (Turkson et al., Mol. Cell. Biol. 18:2545 (1998)). Plasmid pLucTKS3 contains seven copies of Stat3-binding site in TK minimal promoter and its activation specifically depends on Stat3 status in cell environment. pLucTKS3 and pLucSV40 luciferase (a control plasmid lacking Stat3 binding sites) reporter plasmids were transfected into Caov-3 and MDA-MB-435s cell lines using Lipofectamine 2000 reagent. The stable clones, which showed high luciferase activity, were selected for screening Stat3 inhibitors. The selected clones were exposed to Stat3 inhibitors at a final concentration of 20 μM for 48 h and luciferase activity was measured using a Promega Luciferase kit (Madison, Wis.).

Of the 100 small molecules tested, STA-21 showed a remarkable inhibitory effect on Stat3 induced luciferase activity in Caov-3 cloned cells (FIG. 2A). The inhibition of Stat3 activation by STA-21 was further confirmed using MDA-MB-435s cloned cells stably transfected with pLucTKS3 (FIG. 2B). For MDA-MB-435s cloned cells, after the exposure to 20 μM STA-21 for 48 h, luciferase activity was decreased more than five fold (FIG. 2B). This indicates that STA-21 may prevent Stat3 binding to TK promoter region and inhibit luciferase activity in the cloned cells. STA-21 did not affect luciferase activity in the clones transfected with the SV40 luciferase reporter, which does not contain a Stat3 DNA binding site, indicating that STA-21 could not reduce luciferase activity by itself in the absence of a Stat3 DNA binding site (FIG. 2B). As controls, several compounds that did not inhibit Stat3-dependent luciferase activity are shown in FIG. 2A.

### EXAMPLE 3

### Effect of STA-21 on Factors Upstream and Downstream of Stat3

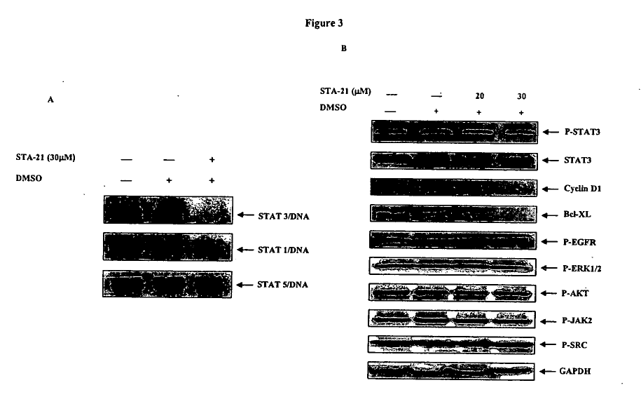

It was next examined whether or not STA-21 could reduce Stat3 DNA binding activity using electrophoretic mobility shift assays as reported (Turkson et al., J. Biol. Chem. 276:45443 (2001)). In MDA-MB-435s breast carcinoma cells with constitutive Stat3 signaling, high Stat3 DNA binding activity was observed (FIG. 3A). In contrast, MCF10A and TERT breast cells without constitutive Stat3 signaling did not show Stat3 DNA binding activity. STA-21 (30 μM) inhibited Stat3 DNA binding activity (FIG. 3A). In MDA-MB-468 breast carcinoma cells with constitutive Stat3 signaling, STA-21 (20 or 30 μM) also inhibited the downstream anti-apoptotic effector Bcl-X_{L} as shown by Western blot analysis (FIG. 3B). Interestingly, the phosphorylation of Stat3 upstream regulators JAK2 (P-JAK2), Src (P-Src), and EGFR (P-EGFR) were not affected by STA-21 (FIG. 3B). Combined with the results of STA-21 inhibition of Stat3 but not Stat1 and Stat5 DNA binding activity in MDA-MB-435s cells (FIG. 3A), the inhibition of Stat3 by STA-21 may be through the direct dysfunction of Stat3 protein and not the inhibition of Stat3 upstream regulators. Meanwhile, STA-21 did not affect the phosphorylation of AKT (P-AKT) or ERK (P-ERK) either (FIG. 3B).

### EXAMPLE 4

### Effect of STA-21 on the Growth and Survival of Breast Carcinoma Cells

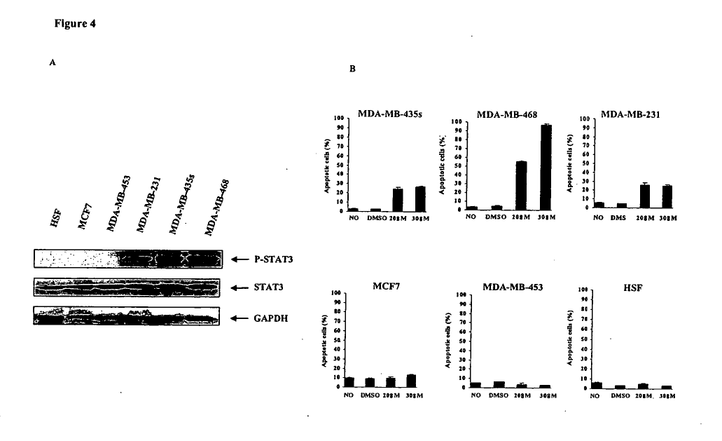

Since STA-21 inhibited Stat3-dependent luciferase and DNA binding activities, it was next examined whether STA-21 inhibited the growth and survival of breast cancer cells with constitutive Stat3 signaling. After cells were exposed to 20 μM or 30 μM of STA-21 for 48 h, STA-21 showed remarkable inhibitory activity on the survival of breast carcinoma cell lines MDA-MB-231, MDA-MB-435s, and MDA-MB-468 that have constitutively activated Stat3 as shown by the accumulation of cells in the sub-G1 phase of the cell cycle indicative of apoptotic cells (FIG. 4B). However, STA-21 had minimal inhibitory effect on MCF7 and MDA-MB-453 breast carcinoma cells and human skin fibroblasts (HSF) that do not have constitutive Stat3 signaling (FIG. 4B). The Stat3 status of the cell lines was confirmed by measurement of phosphorylated Stat3 (FIG. 4A). Combined with data from cell viability assays using MTT, STA-21 demonstrated strong potential to inhibit the growth and survival of breast cancer cells that contain constitutively active Stat3.

### EXAMPLE 5

### Effect of STA-21 on Stat3 Translocation and Dimerization

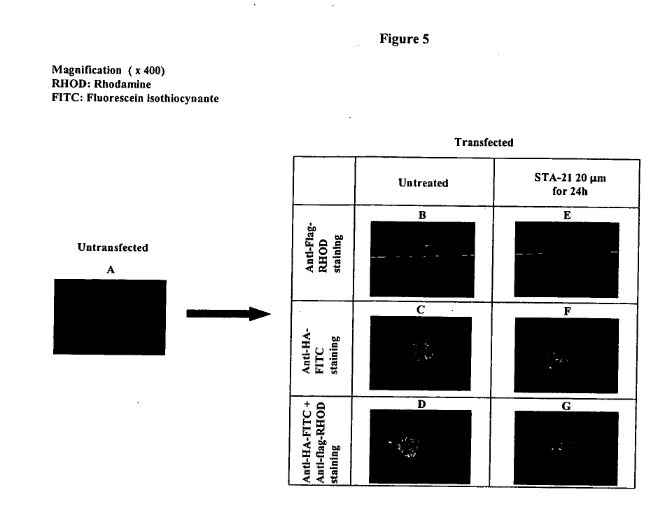

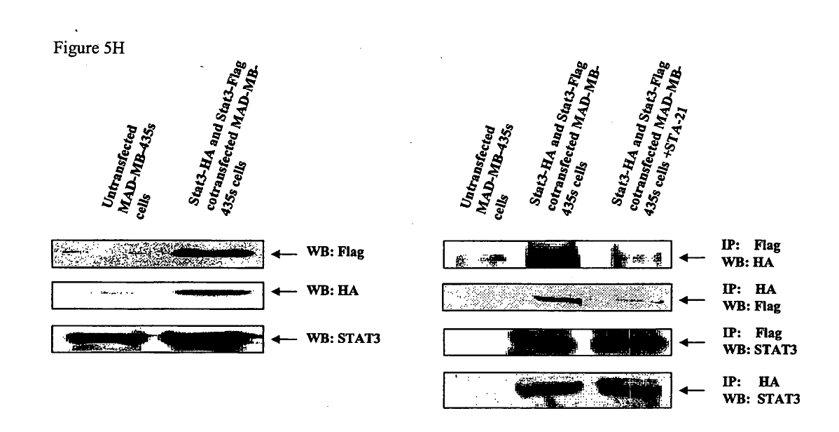

The plasmids pCMV-Stat3-Flag and pCMV-Stat3-HA for expression of Stat3-Flag and Stat3-HA tagged proteins were cotransfected into MDA-MB-435s breast carcinoma cells. The transfected cells were exposed to STA-21 (20 μM) for 24 h, then fixed with 100% methanol for 30 min at −20° C. Following 3× washes using phosphate-buffered saline (PBS), anti-HA (rabbit, Santa Cruz Biotechnology) and/or anti-Flag (mouse, Sigma) antibodies were added to the cells and the cells incubated for 1 h at 37° C. The cells were washed 3× with PBS buffer, and secondary antibodies for anti-rabbit IgG-fluorescein isothiocyanate (FITC) or/and anti-mouse IgG-Rhodamine (RHOD) were added and the cells incubated for 1 h. Following 3× washes with PBS, the cells were observed using a fluorescence microscope. The results showed that the nuclear translocation of Stat3-Flag and Stat3-HA proteins was blocked by STA-21 (FIGS. 5A-5G). From FITC (green) and Rhodamine (red) separate staining, STA-21 inhibited Stat3-Flag or Stat3-HA nuclear translocation as shown in FIGS. 5E and 5F. In the cells without STA-21 treatment, strong orange staining was observed in the nucleus using combined FITC and Rhodamine staining, indicating that Stat3-Flag and Stat3-HA tagged proteins co-localized into the nucleus and the two colors merged to become orange (FIG. 5D). However, when the cells were treated with 20 μM of STA-21, much weaker orange staining appeared throughout the entire cell (FIG. 5G), suggesting that STA-21 blocked nuclear translocation of Stat3-Flag and Stat3-HA proteins. As a control, DMSO using as solvent showed no effect on the cells.

The effect of STA-21 on Stat3 in vivo dimerization in breast cancer MDA-MB-435s cells was also investigated. After the exposure to 20 μM STA-21 for 24 h, the cells were harvested and the lysates from the cells expressing Stat3-Flag and Stat3-HA tagged proteins were immunoprecipitated with an anti-Flag or anti-HA antibody, respectively. The immunoprecipitated reaction mixtures were resolved on a 10% SDS-PAGE and immunoblotted with an anti-HA, anti-Flag, or anti-Stat3 antibody, respectively. The results showed that STA-21 abrogated Stat3 dimerization between Stat3-Flag and Stat3-HA proteins in the MDA-MB-435 cancer cells (FIG. 5H).

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

What is claimed is:

1. A method of inhibiting Stat3 activity in a cell, comprising contacting the cell with STA-21 or a derivative, analog, prodrug, or pharmaceutically acceptable salt thereof.

2. A method of inhibiting the growth of a cell having elevated Stat3 activity, comprising contacting the cell with STA-21 or a derivative, analog, prodrug, or pharmaceutically acceptable salt thereof.

3. A method of treating, ameliorating, or preventing a disorder associated with elevated Stat3 activity in an animal, comprising administering to said animal a therapeutically effective amount of STA-21 or a derivative, analog, prodrug, or pharmaceutically acceptable salt thereof.

4. A method of inducing apoptosis and/or cell cycle arrest in a cell, comprising contacting the cell with STA-21 or a derivative, analog, prodrug, or pharmaceutically acceptable salt thereof.

5. A method of rendering a cell sensitive to an inducer of apoptosis, comprising contacting the cell with STA-21 or a derivative, analog, prodrug, or pharmaceutically acceptable salt thereof.

6. The method of claim 5, further comprising contacting the cell with an inducer of apoptosis.

7. The method of claim 6, wherein said inducer of apoptosis is a chemotherapeutic agent.

8. The method of claim 6, wherein said inducer of apoptosis is radiation.

9. A method of treating, ameliorating, or preventing a disorder responsive to the induction of apoptosis in an animal, comprising administering to said animal a therapeutically effective amount of STA-21 or a derivative, analog, prodrug, or pharmaceutically acceptable salt thereof.

10. The method of claim 9, further comprising administering an inducer of apoptosis.

11. The method of claim 10, wherein said inducer of apoptosis is a chemotherapeutic agent.

12. The method of claim 10, wherein said inducer of apoptosis is radiation.

13. The method of claim 9, wherein said disorder responsive to the induction of apoptosis is a hyperproliferative disease.

14. The method of claim 13, wherein said hyperproliferative disease is cancer.

15. The method of claim 14, wherein said cancer is breast cancer or ovarian cancer.

16. The method of claim 13, wherein said hyperproliferative disease is psoriasis.

17. The method of claim 10, wherein said STA-21 or a derivative, analog, prodrug, or pharmaceutically acceptable salt thereof is administered prior to said inducer of apoptosis.

18. The method of claim 10, wherein said STA-21 or a derivative, analog, prodrug, or pharmaceutically acceptable salt thereof is administered after said inducer of apoptosis.

19. The method of claim 10, wherein said STA-21 or a derivative, analog, prodrug, or pharmaceutically acceptable salt thereof is administered concurrently with said inducer of apoptosis.

20. The method of any one of claims 1-5 and 9, wherein said STA-21 or a derivative, analog, prodrug, or pharmaceutically acceptable salt thereof is selected from the group consisting of compounds 1 (STA-21), 2, and 3.

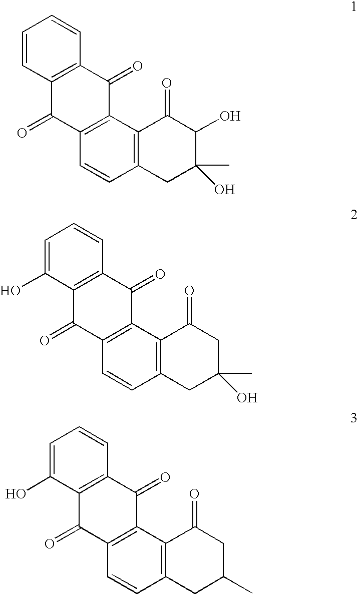

21. A pharmaceutical composition comprising STA-21 or a derivative, analog, prodrug, or pharmaceutically acceptable salt thereof and a pharmaceutically acceptable carrier.

22. A kit comprising STA-21 or a derivative, analog, prodrug, or pharmaceutically acceptable salt thereof and instructions for administering said compound to an animal.

23. The kit of claim 22, further comprising an inducer of apoptosis.

24. The kit of claim 23, wherein said inducer of apoptosis is a chemotherapeutic agent.

25. The kit of claim 22, wherein said instructions are for administering said compound to an animal having a hyperproliferative disease.

26. The kit of claim 25, wherein said hyperproliferative disease is cancer.

27. The kit of claim 26, wherein said cancer is breast cancer or ovarian cancer.

28. The kit of claim 25, wherein said hyperproliferative disease is psoriasis.
